(19) United States
(12) Patent Application Publication
(10) Pub. No.: US 2024/0299266 A1
(43) Pub. Date: Sep. 12, 2024

(54) FRAGRANCE COMPOSITIONS WITH MOISTURIZING BENEFITS

(71) Applicant: Firmenich SA, Satigny (CH)

(72) Inventors: Vera TCHAKALOVA, Satigny (CH); Laura MESMIN, Satigny (CH)

(21) Appl. No.: 18/693,110

(22) PCT Filed: Nov. 1, 2022
(86) PCT No.: PCT/EP22/80449

§ 371 Date: Mar. 18, 2024

(30) Foreign Application Priority Data
Nov. 3, 2021 (EP) 21206123.8

(51) Int. Cl. A61K 8/33; A61K 8/31; A61K 8/36; A61K 8/37; A61K 8/49; A61Q 19/00; C11B 9/00; G01N 33/50

(52) U.S. Cl. CPC A61K 8/33; A61K 8/31; A61K 8/36; A61K 8/37; A61K 8/4973; A61Q 19/007; C11B 9/003; C11B 9/0034; C11B 9/0042; C11B 9/0076; C11B 9/0084; G01N 33/5082

(57) ABSTRACT

Described herein is a method of using a composition including a fragrance component, where the fragrance component is present in an amount of from about 0.1% to 96% relative to the total weight of the composition where the fragrance component comprises one or more perfume ingredients selected from a specific list. The method includes using the composition as a skin moisturizer.

### INTRODUCTION

The invention relates to the field of fragrance design.

Fragrance design can be defined as the selection of at least one fragrant ingredient to form a composition intended to provide a targeted fragrance. Fragrance design is most notably known in the field of perfumery and is performed by perfume designers.

The evaluation of a fragrance is typically based on fragrance performance metrics and fragrance hedonics alone. Several metrics are used today, such as the detectability of the fragrance by a human nose for example. Typically, such metrics fail to account for additional benefits of fragrances, such as cosmetic benefits for example.

As such metrics lack for evaluating the performance of a fragrance according to said secondary benefits, no accurate performance predictor in these regards may be obtained. This leads to much wasted time in laboratories from inefficient but currently obligatory trial and error approaches.

One such additional benefit is skin moisturizing. Moisturizing of the skin can be achieved by two different mechanisms: 1) providing hydration via hydrophilic compounds called humectants such as the glycerol; and 2) preventing the water loss by hydrophobic compounds called moisturizers such as mineral oil to protect the skin.

Until know, to our knowledge, the fragrances were used mainly for their hedonic benefice. However, in our days the consumers need more than hedonic performance, they are looking for wellbeing benefits such as cosmetic or hygiene benefits. Therefore, there is a need to create fragrances, which are able to deliver more than the pleasant smell. The present invention is satisfying this consumer requirement claiming fragrances with secondary cosmetic benefits, defining hydrating fragrance term and a method appropriate for the systematic evaluation of skin hydration properties of fragrances and the components of fragrance compositions.

### SUMMARY OF THE INVENTION

A first aspect of the invention provides the use of a composition comprising a fragrance component, said fragrance component is present in an amount of from about 0.1% to 96% relative to the total weight of the composition wherein the fragrance component comprises one or more perfume ingredients selected from the group comprising: benzyl acetate, 1-oxa-12-cyclohexadecen-2-one (a)+1-oxa-13-cyclohexadecen-2-one, (+)-methyl (1s)-trans-3-oxo-2-pentyl-1-cycloheptaneacetate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one, (3ar,5as,9as,9br)-3a,6,6,9a-tetramethyldodecahydronaphtho[2,1-b]furan, oxacyclohexadecan-2-one and 1-(5,5-dimethyl-1-cyclohexen-1-yl)-4-penten-1-one, (+−)-8-methoxycedrane, 1-methyl-4-(1-methylethenyl)-cyclohexene, (3RS,3aRS,6SR,7aSR)-3,6-dimethylhexahydro-1-benzofuran-2(3H)-one (A)+(3RS,3aSR,6RS,7aRS)-3,6-dimethylhexahydro-1-benzofuran-2(3H)-one (B), (3aR,5aS,9aS,9bR)-3a,6,6,9a-tetramethyldodecahydronaphtho[2,1-b]furan, (+)-2-{(1S)-1-[(1R)-3,3-dimethylcyclohexyl]ethoxy}-2-methylpropyl propionate, almond oil, mineral oil, petroleum Jelly, as a skin moisturizer.

An embodiment of the invention is wherein the fragrance component comprises benzyl acetate, (+−)-3,7-dimethyl-6-octen-1-ol, (+−)-2,6-dimethyl-7-octen-2-ol (+−)-tetrahydro-2-isobutyl-4-methyl-4(2h)-pyranol, (e)-3,7-dimethyl-2,6-octadien-1-ol, 1-oxa-12-cyclohexadecen-2-one (a)+1-oxa-13-cyclohexadecen-2-one, (+)-methyl (1s)-trans-3-oxo-2-pentyl-1-cyclopentane acetate, (+)-2-{(1s)-1-[(1r)-3,3-dimethylcyclohexyl]ethoxy}-2-methylpropyl propionate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one and 2-phenyl ethanol.

A further embodiment of the invention is wherein the fragrance component comprises benzyl acetate, (+−)-2,6-dimethyl-7-octen-2-ol, 4-cyclohexyl-2-methyl-2-butanol, (+)-2-{(1s)-1-[(1r)-3,3-dimethylcyclohexyl]ethoxy}-2-methylpropyl propionate, 1-((2rs,3rs)-2,3,8,8-tetramethyl-1,2,3,4,5,6,7,8-octahydronaphthalen-2-yl)ethenone, (3ar,5as,9as,9br)-3a,6,6,9a-tetramethyldodecahydronaphtho[2,1-b]furan oxacyclohexadecan-2-one and 1-(5,5-dimethyl-1-cyclohexen-1-yl)-4-penten-1-one

A further embodiment of the invention is wherein the fragrance component comprises 1-oxa-12-cyclohexadecen-2-one (a)+1-oxa-13-cyclohexadecen-2-one, (+)-methyl (1s)-trans-3-oxo-2-pentyl-1-cyclopentane acetate, (+−)-(4e)-3-methyl-4-cyclopentadecen-1-one, 3ar,5as, 9as,9br)-3a,6,6,9a-tetramethyldodecahydronaphtho[2,1-b]furan and oxacyclohexadecan-2-one.

A further embodiment of the invention is wherein the composition further comprises a perfumery carrier and/or a perfuming co-ingredient and/or a perfumery adjuvant.

A further aspect of the invention is the use of a perfumed consumer product comprising a composition as defined in any one of the previous claims as a skin moisturizer.

An embodiment of the invention is wherein the perfumed consumer product is a perfume, a body-care product, a cosmetic preparation or a skin-care product.

A further embodiment of the invention is wherein the perfumery consumer product is a fine perfume, a splash or eau de parfum, a cologne, a shave or after-shave lotion, a shampoo, a hair conditioner, a coloring preparation, a color care product, a hair shaping product, a dental care product, a disinfectant, an intimate care product, a hair spray, a vanishing cream, a deodorant or antiperspirant, a hair remover, a tanning or sun product, a nail product, a skin cleansing, a makeup, a perfumed soap, a shower or bath mousse, an oil or a gel, or a foot/hand care products a hygiene product.

A further aspect of the invention provides a method to confer, enhance, improve or modify the skin moisturizing properties of a perfuming composition or of a perfumed article, which method comprises adding to said composition or article an effective amount of a composition of the invention.

A further aspect of the invention provides a method to confer, enhance, improve or modify skin moisture which method comprises applying said effective amount of a composition or consumer product of the invention to the skin of a subject in need thereof.

A further aspect of the invention provides a skin replicating surface comprising (i) a hydrogen membrane layer comprising a water permeable substrate supporting a biopolymer, and (ii) a stratum corneum mimic layer.

### DESCRIPTION OF THE FIGURES

FIG. 1: Reproducibility of the measured TEWL of the reference system (Relative humidity: 27%, Temperature: 24° C.).

### DETAILED DESCRIPTION OF THE INVENTION

This description is not exhaustive, as each feature of one embodiment may be combined with any other feature of any other embodiment in an advantageous manner.

It should be noted at this point that the figures are not to scale.

The present invention relates to the use of a composition comprising a fragrance component, said fragrance component is present in an amount of from about 0.1% to 96% relative to the total weight of the composition wherein the fragrance component comprises one or more perfume ingredients selected from the group comprising: benzyl acetate, 1-oxa-12-cyclohexadecen-2-one (a)+1-oxa-13-cyclohexadecen-2-one, (+)-methyl (1s)-trans-3-oxo-2-pentyl-1-cycloheptaneacetate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one, (3ar,5as,9as,9br)-3a,6,6,9a-tetramethyldodecahydronaphtho[2,1-b]furan, oxacyclohexadecan-2-one and 1-(5,5-dimethyl-1-cyclohexen-1-yl)-4-penten-1-one, (+−)-8-methoxycedrane, 1-methyl-4-(1-methylethenyl)-cyclohexene, (3RS,3aRS,6SR,7aSR)-3,6-dimethylhexahydro-1-(3RS,3aSR,6RS,7aRS)-3,6-dimethylhexahydro-1-benzofuran-2(3H)-one (A)+benzofuran-2(3H)-one (B), (3aR,5aS,9aS,9bR)-3a,6,6,9a-tetramethyldodecahydronaphtho[2,1-b]furan, (+)-2-{(1S)-1-[(1R)-3,3-dimethylcyclohexyl]ethoxy}-2-methylpropyl propionate, almond oil, mineral oil, petroleum jelly, as a skin moisturizer.

As used herein, the terms “fragrant component” designates a perfuming ingredient, a flavor ingredient, a perfumery carrier, a flavor carrier, a perfumery adjuvant, a flavor adjuvant, a perfumery modulator, flavor modulator. Preferably, such a fragrance component is volatile. Such an ingredient may be a natural ingredient.

By “perfuming ingredient” it is meant here a compound, which is used in a perfuming preparation or a composition to impart a hedonic effect. In other words such an ingredient, to be considered as being a perfuming one, must be recognized by a person skilled in the art as being able to impart or modify the odor of a composition.

As used herein, the term “formula” designates a liquid, solid or gaseous assembly of at least one volatile molecule.

As used herein, the term “fragrance” refers to the olfactory perception resulting from the sum of odorant receptor activation, enhancement, and inhibition by at least one fragrant chemical compound.

The nature and type of the perfuming ingredient do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said fragrance component co-ingredients can be of natural or synthetic origin. Said fragrance component are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said fragrance component may also be compounds known to release in a controlled manner various types of perfuming ingredients also known as properfume or profragrance.

The present inventors have investigated the moisturizing potential of perfume ingredients. To perform this assessment they used a model called a skin replicating surface, and measured the transepidermal water loss (TEWL) before and after the deposit of a sample of the perfume ingredient. This is measure of the moisturizing activity of the perfume ingredient.

The said moisturizing activity is function of the water evaporation rate of the skin replicating surface before and after the deposition of the fragrance component.

The moisturizing activity of a fragrance component may be calculated as follows.
a step of controlled deposition of a fragrance component upon an skin replicating surface,
a step of measurement of a quantity of evaporated water from the skin replicating surface or of remaining water on the skin replicating surface after the deposition of the fragrance component at different measurement times,
a step of reduction of the water evaporation rate calculation depending on the measured evaporated or remaining water quantities measured,
a step of moisturizing activity calculation as a function of the water evaporation rate measured.

The step of controlled deposition is performed, for example, by the transfer upon the skin replicating surface of a predetermined quantity of fragrance component set to spread over a predetermined surface. Such predetermination allows for the comparison of results as evaporation is dependent on the surface area wetted by the fragrance component in contact with the ambient environment. The more parameters are set and predetermined, the more accurate the water evaporation rate measurement is.

The transfer of the quantity of fragrance component can be performed using any known means to transfer liquids, preferably in small quantity, such as a pipette. Such a transfer can be performed manually or in an automatic manner.

The fragrance component considered can be in the form of a liquid or in the form of a solid diluted into a liquid, or in a form of a gel or wax.

Rather than being adapted to single-fragrance component analysis, the method may be used upon fragrance compositions or mixtures, creating a database of compositions or mixture secondary benefit performance indicators.

This step of controlled deposition is preferably performed at a controlled temperature and humidity throughout the evaporation quantity measurement.

Water evaporation rates or water content in the skin (in-vitro or in-vivo) are preferably measured in pseudo-equilibrium conditions: controlled temperature, air flow and rate, humidity to mimic a closed thermodynamic system.

The step of measurement is performed, for example, using a Franz cell, whereby the skin replicating surface is placed within said cell, acting as the membrane, and the fragrance component sample to be analyzed is placed upon said surface. Measurement can typically be performed at the top of the Franz cell, above the membrane, which is different from the usual use of the Franz cell in which the measurements are made via a sampling port located below the membrane.

In other variants, any other type of measurement device may be used.

During this step of measurement, the water loss from within the skin or skin model (in-vivo or in-vitro) to the external atmosphere or the water content remaining the skin or skin model is measured. In other embodiments, both are measured. Such measurements are preferably performed as function of time.

Such a measurement may be performed by a water evaporation quantity or water content measurement device (vapometer or moisturemeter) to produce values representative of Trans-Epidermal Water Loss (“TEWL”) or the water content.

Such measurements may be impacted by:
temperature,
surface of exposition upon the human skin mimic,
quantity of compound deposited,
air flow volume and
humidity
rate of evaporation of the deposited fragrance component
rate of penetration of the deposited fragrance component.

The step of water evaporation rate calculation may be performed by running a computer software upon a computing device. Such a computing device may be unitary with a water evaporation quantity measurement device, for example. In other embodiments, such a computing device may be a computer or server associated to the water evaporation quantity measurement device. The calculated water evaporation rate may be such that said water evaporation rate is obtained by dividing the quantity of water evaporated measured by a value representative of a duration of evaporation. Depending on the characteristics of the system, such a duration may be 30 minutes, one hour or other such intervals. In particular embodiments, several water evaporation rates are computed for on chemical compound for different durations from fragrance component deposition.

In other embodiments, the calculated water evaporation rate may be such that said water evaporation rate is obtained by subtracting the quantity of remaining water from the initial quantity of water deposited and dividing the resulting quantity of water evaporated measured by a value representative of a duration of evaporation.

Such a water evaporation rate may be measured in quantity (absolute or relative) to be processed to produce a water evaporation rate. For example, a value of water evaporation rate may be given for a skin mimicking surface after deposition of a chemical compound losing 5% of the original deposited water quantity, due to evaporation, over 30 minutes. The water evaporation rate (g/m² h) can be calculated from the increase of relative humidity in function of time. Ambient temperature and humidity can be recorded using an external room sensor to account for environmental offset of the pending measurement.

The method may further comprise a step of water evaporation reference quantity measurement a step of water evaporation reference rate calculation. Both these steps may be performed similarly to, or during, the step of measurement and the step of calculation disclosed above. In these steps, water replaces the fragrant fragrance component, allowing for the definition of a reference value for evaporation rates comparison.

By measuring the moisturizing potential of a range of different perfume ingredients, the present inventors determined that the following perfumes have a suitable moisturizing potential: benzyl acetate, 1-oxa-12-cyclohexadecen-2-one (a)+1-oxa-13-cyclohexadecen-2-one, (+)-methyl (1s)-trans-3-oxo-2-pentyl-1-cycloheptaneacetate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one, (3ar,5as,9as,9br)-3a,6,6,9a-tetramethyldodecahydronaphtho[2,1-b]furan, oxacyclohexadecan-2-one and 1-(5,5-dimethyl-1-cyclohexen-1-yl)-4-penten-1-one, (+−)-8-methoxycedrane, 1-methyl-4-(1-methylethenyl)-cyclohexene, (3RS,3aRS,6SR,7aSR)-3,6-dimethylhexahydro-1-benzofuran-2(3H)-one (A)+(3RS,3aSR,6RS,7aRS)-3,6-dimethylhexahydro-1-benzofuran-2(3H)-one (B), (3aR,5aS,9aS,9bR)-3a,6,6,9a-tetramethyldodecahydronaphtho[2,1-b]furan, (+)-2-{(1S)-1-[(1R)-3,3-dimethylcyclohexyl]ethoxy}-2-methylpropyl propionate, almond oil, mineral oil, petroleum Jelly, as a skin moisturizer.

From this information the inventors then prepared a number of different moisturizing agent accords.

In one embodiment the fragrance component comprises benzyl acetate, (+−)-3,7-dimethyl-6-octen-1-ol, (+−)-2,6-dimethyl-7-octen-2-ol (+−)-tetrahydro-2-isobutyl-4-methyl-4(2h)-pyranol, (e)-3,7-dimethyl-2,6-octadien-1-ol, 1-oxa-12-cyclohexadecen-2-one (a)+1-oxa-13-cyclohexadecen-2-one, (+)-methyl (1s)-trans-3-oxo-2-pentyl-1-cyclopentane acetate, (+)-2-{(1s)-1-[(1r)-3,3-dimethylcyclohexyl]ethoxy}-2-methylpropyl propionate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one and 2-phenyl ethanol.

In another embodiment the fragrance component comprises benzyl acetate, (+−)-2,6-dimethyl-7-octen-2-ol, 4-cyclohexyl-2-methyl-2-butanol, (+)-2-{(1s)-1-[(1r)-3,3-dimethylcyclohexyl]ethoxy}-2-methylpropyl propionate, 1-((2rs,3rs)-2,3,8,8-tetramethyl-1,2,3,4,5,6,7,8-octahydronaphthalen-2-yl)ethenone, (3ar,5as,9as,9br)-3a,6,6,9a-tetramethyldodecahydronaphtho[2,1-b]furan oxacyclohexadecan-2-one and 1-(5,5-dimethyl-1-cyclohexen-1-yl)-4-penten-1-one

In another embodiment the fragrance component comprises 1-oxa-12-cyclohexadecen-2-one (a)+1-oxa-13-cyclohexadecen-2-one, (+)-methyl (1s)-trans-3-oxo-2-pentyl-1-cyclopentane acetate, (+−)-(4e)-3-methyl-4-cyclopentadecen-1-one, 3ar,5as,9as,9br)-3a,6,6,9a-tetramethyldodecahydronaphtho[2,1-b]furan and oxacyclohexadecan-2-one.

Examples of further components which may be used include mineral oil, vegetable oils, emollients, waxes, butters, esters such as isopropyl myristate, film formers such as biopolymers, synthetic polymers, thickeners, particles such as mica, TiO2, and other such components.

A further embodiment of the composition of the invention is wherein the composition further comprises a perfumery carrier and/or a perfuming co-ingredient and/or a perfumery adjuvant.

By “perfumery carrier” it is meant here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples, solvents such as the diols (butylene-, propylene-, pentylene-, hexylene-, octane-, etc), glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethano, tri-ethyl citrate or mixtures thereof, which are the most commonly used. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. The use of solid carriers is of current use in the art and a person skilled in the art knows how to reach the desired effect. However, by way of non-limiting examples of solid carriers, one may cite absorbing gums or polymers or inorganic materials, such as porous polymers, cyclodextrins, wood based materials, organic or inorganic gels, clays, gypsum talc or zeolites, or cosmetically active non-odorous molecules.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

By “perfumery adjuvant”, it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof.

By “perfumery modulator”, it is understood here an agent having the capacity to affect the manner in which the odour, and in particular the evaporation rate and intensity, of the compositions incorporating said modulator can be perceived by an observer or user thereof, over time, as compared to the same perception in the absence of the modulator. Perfumery modulators are also known as fixative. In particular, the modulator allows prolonging the time during which their fragrance is perceived. Non-limiting examples of suitable modulators may include methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, emollients, monoglycerol esters (laurate, myristate, palmitate, stearate, . . . ), phytantriol, a profragrance, cyclodextrin, an encapsulation, and a combination thereof.

Suitable liquid carriers include, for instance, an emulsifying system, i.e. a solvent and a surfactant system, or a solvent commonly used in flavors. A detailed description of the nature and type of solvents commonly used in flavor cannot be exhaustive. Suitable solvents used in flavor include, for instance, propylene glycol, triacetine, caprylic/capric triglyceride (Neobee®), triethyl citrate, benzylic alcohol, ethanol, isopropanol, citrus terpenes, vegetable oils such as Linseed oil, sunflower oil or coconut oil, glycerol.

Suitable solid carriers include, for instance, absorbing gums or polymers, or even encapsulating materials. Examples of such materials may comprise wall-forming and plasticizing materials, such as mono, di- or polysaccharides, natural or modified starches, hydrocolloids, cellulose derivatives, polyvinyl acetates, polyvinylalcohols, xanthan gum, alginate salts and acids, carageenan, arabic gum, acacia gum or yet the materials cited in reference texts such as H. Scherz, Hydrokolloid: Stabilisatoren, Dickungs-und Geliermittel in Lebensmitteln, Band 2 der Schriftenreihe Lebensmittelchemie, Lebensmittelqualität, Behr's VerlagGmbh & Co., Hamburg, 1996. Encapsulation is a well-known process to a person skilled in the art, and may be performed, for instance, using techniques such as spray-drying, agglomeration, extrusion, coating, plating, coacervation and the like.

As used herein, the term “formula” designates a liquid, solid or gaseous assembly of at least one volatile molecule.

The composition of the invention is for use in as a skin moisturizer. By “moisturizer” we mean that the composition of the invention reduces water loss from the skin.

A further aspect of the invention provides a method of improving skin moisture comprising applying a composition of the invention to the skin.

A further aspect of the invention provides a perfumed consumer product comprising a composition of the invention.

The invention's composition can also be advantageously used in all the fields of modern perfumery, i.e. fine or functional perfumery, to positively impart or modify the odor of a consumer product into which said composition is added. Consequently, another object of the present invention consists of a perfumed consumer product comprising, as a perfuming ingredient, the invention's composition, as defined above.

For the sake of clarity, “perfumed consumer product” is meant to designate a consumer product which deliver at least a pleasant perfuming effect to the surface or space to which it is applied (e.g. skin, hair). In other words, a perfumed consumer product according to the invention is a perfumed consumer product which comprises a functional formulation, as well as optionally additional benefit agents, corresponding to the desired consumer product and an olfactive effective amount of at least one invention's compound. For the sake of clarity, said perfumed consumer product is a non-edible product.

The invention's composition can be added as such or as part of an invention's perfuming composition.

The nature and type of the constituents of the perfumed consumer product do not warrant a more detailed description here, which in any case would not be exhaustive, the skilled person being able to select them on the basis of his general knowledge and according to the nature and the desired effect of said product.

An embodiment of the invention is wherein the perfumery consumer product is a perfume, a body-care product, a cosmetic preparation or a skin-care product. Preferably the perfumery consumer product is a fine perfume, a splash or eau de parfum, a cologne, a shave or after-shave lotion, a shampoo, a coloring preparation, a color care product, a hair shaping product, a dental care product, a disinfectant, an intimate care product, a hair spray, a vanishing cream, a deodorant or antiperspirant, a hair remover, a tanning or sun product, a nail product, a skin cleansing, a makeup, a perfumed soap, a shower or bath mousse, an oil or a gel, or a foot/hand care products a hygiene product.

Preferred consumer products include skin cleaning, skin care and skin deodorizing products, including brands well known in the market place from major consumer product companies. Such products include soap bars, shower gels and also liquid hand soap products, as well as roll-on and stick format deodorant formulations.

Some of the above-mentioned perfumed consumer products may represent an aggressive medium for the invention's composition, so that it may be necessary to protect the latter from premature decomposition, for example by encapsulation or by chemically binding it to another chemical which is suitable to release the invention's composition upon a suitable external stimulus, such as an enzyme, light, heat or a change of pH.

The proportions in which the composition according to the invention can be incorporated into the various aforementioned products or compositions vary within a wide range of values. These values are dependent on the nature of the article to be perfumed and on the desired organoleptic effect as well as on the nature of the co-ingredients in a given base when the invention's composition according to the invention are mixed with perfuming co-ingredients, solvents or additives commonly used in the art.

When the invention composition are incorporated into perfumed consumer products, other than fine perfumery and air freshener where in general the perfuming composition is the essential constituent of a consumer product, 0.01% to 5% by weight of the invention's composition can be used, percentage being relative to the weight of the article.

A further aspect of the invention provides a method to confer, enhance, improve or modify the skin moisturizing properties of a perfuming composition or of a perfumed article, which method comprises adding to said composition or article an effective amount of a composition as defined in any of the previous claims.

A further aspect of the invention provides a skin replicating surface comprising (i) a hydrogel membrane layer comprising a water permeable substrate supporting a biopolymer, and (ii) a stratum corneum mimic layer.

In the context of this invention, a “skin replicating surface” designates any surface presenting physicochemical properties similar to the human skin, including human skin itself. Simple embodiments of such an artificial surface may focus on a mimicking a limited number of physicochemical properties, such as thickness, chemical reactivity, or visco-elasticity of the human skin. Other embodiments may focus on a particular element of the human skin, such as the epidermis, the dermis layer and/or stratum corneum of the human skin.

Such a surface may be as simple as a glass surface or as complex as a multi-layer model. The closer such surface is to replicating actual properties of the human skin, the better the quality of database construction and the better the quality of downstream prediction.

In the context of this invention, an “skin replicating surface” designates any surface presenting physicochemical properties similar to the human skin, including human skin itself. Simple embodiments of such an artificial surface may focus on a mimicking a limited number of physicochemical properties, such as thickness, chemical reactivity, or visco-elasticity of the human skin. Other embodiments may focus on a particular element of the human skin, such as the epidermis, the dermis layer and/or stratum corneum of the human skin.

### EXAMPLES

### Example 1: An In Vitro Skin Model and Method for Assessment

An in-vitro method for ingredients and formulations cosmetic benefits assessment was developed.

The skin replicating surface comprising (i) a hydrogel membrane layer comprising a water permeable substrate supporting a biopolymer, and (ii) a stratum corneum mimic layer.

The hydrogel membrane composition should include a knitted cotton piece topped by a hydrogel containing biopolymer (gelatin) able to form a hydrogel with water and glycerol.

The stratum corneum mimic layer can be a Vitro-Corneum substrate (product of IMS inc.)

Before measurements, the hydrogel membrane and the Vitro-Corneum substrate should be separately hydrated in a hydration chamber and then, assembled. The Vitro-Corneum substrate is made of proteins and lipids and mimics the thickness, the visco-elasticity, the chemical reactivity and the surface properties of human stratum corneum. The hydrogel allows to keep the Vitro-Corneum substrate hydrated.

### An Example of how to Obtain the In-Vitro Skin Model and Composition

Hydrogel is prepared by mixing gelatin, glycerol and water at a water bath in the following composition. Preferably around 50% of the hydrogel is glycerol and around 5% is gelatin completed to 100% with water. This is added to a supporting surface and allowed to set.

Both part of the artificial skin model needs to be hydrated before being assembled. Hydrogel membranes and the Vitro-Corneum substrate (2×2 cm) are placed into an hydration chamber containing the glycerol/water wetting solution. Hydrogel membranes are hydrated and Vitro-Corneum substrates are hydrated. When the hydration is complete, the Vitro-Corneum substrate is deposited on the hydrogel. The final skin model is now ready for experiments.

### TEWL Measurements

The TEWL (Trans-Epidermal Water Loss) measurements were performed with a Vapometer and Moisturemeter, product of Delfin Technologies (Finland).

Hydrated artificial skin membrane is placed on the top of a Franz Cell, 50 μL of the product to be tested is applied on the membrane and spread manually with a finger protected by a glove. The Vapometer measures the water evaporation rate thanks to sensitive humidity sensors mounted in a cylindrical measurement chamber at the core of the device. During the measurement, the chamber is closed by the support and unaffected by ambient airflows. The water evaporation rate (g/m² h) is calculated from the increase of relative humidity in function of time. Ambient temperature and humidity are recorded using an external room sensor.

For our TEWL measurement method, each experiment lasts one hour, and several measurements were taken at different time points during this period.

### System of Reference

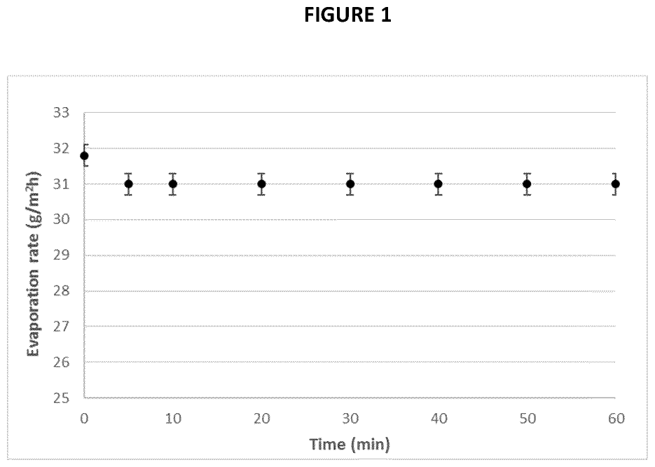

All the experiments were compared to a reference system: hydrated artificial skin model, pre-treatment, without raw material application. The evaporation rate of water through the hydrated membrane is stable and reproducible for one hour (FIG. 1); it is equivalent to 31 g/m² h (relative humidity: 27%, temperature: 24° C.). For each experiment, the evaporation rate was measured before the ingredient application and the TEWL results were normalized by its value. With the normalization, the reference corresponds to 100% of water evaporation.

### Example 2: Cosmetic Benefits of Perfumery Ingredients

Cosmetic benefits and more specifically moisturizing was determined for some perfumery ingredients using the in-vitro method developed for this purpose (Example 1).

The following are perfume ingredients which have moisturizing effects: (+−)-8-methoxycedrane, 1-methyl-4-(1-methylethenyl)-cyclohexene, Oxacyclohexadecan-2-one, Cycloheptaneacetic acid, 3-oxo-2-pentyl-, 2-{(1S)-1-[(1R)-3,3-dimethylcyclohexyl]ethoxy}-2-oxoethyl propionate, Benzyl acetate, (3RS,3aRS,6SR,7aSR)-3,6-dimethylhexahydro-1-benzofuran-2(3H)-one (A)+(3RS,3aSR,6RS,7aRS)-3,6-dimethylhexahydro-1-benzofuran-2(3H)-one (B), 2-{(1S)-1-[(1R)-3,3-dimethylcyclohexyl]ethoxy}-2-oxoethyl propionate

### Example 3. Cosmetic Benefits of Fragrance Compositions

Based on the methods presented herein fragrance compositions with and without moisturizing benefits were created.

moisturizing fragrance: (3ar,5as,9as,9br)-3a,6,6,9a-tetramethyldodecahydronaphtho[2,1-b]furan, oxacyclohexadecan-2-one, 1-oxa-12-cyclohexadecen-2-one (a)+1-oxa-13-cyclohexadecen-2-one, (+)-methyl (1s)-trans-3-oxo-2-pentyl-1-cycloheptaneacetate, (+)-2-{(1s)-1-[(1r)-3,3-dimethylcyclohexyl]ethoxy}-2-methylpropyl propionate, (+−)-(4e)-3-methyl-4-cyclopentadecen-1-one.

non-moisturizing fragrance: benzyl acetate, (+−)-3,7-dimethyl-6-octen-1-ol, +−)-2,6-dimethyl-7-octen-2-ol, (+−)-tetrahydro-2-isobutyl-4-methyl-4(2h)-pyranol, (e)-3,7-dimethyl-2,6-octadien-1-ol, 1-oxa-12-cyclohexadecen-2-one (a)+1-oxa-13-cyclohexadecen-2-one, (+)-methyl (1s)-trans-3-oxo-2-pentyl-1-cycloheptaneacetate, (+)-2-{(1s)-1-[(1r)-3,3-dimethylcyclohexyl]ethoxy}-2-methylpropyl propionate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one, 2-phenyl ethanol.

### Example 4: Moisturizing of Cosmetic Agents

Moisturizing of well-known cosmetic agents: dimethicone, mineral oil, lanolin, petroleum jelly, was measured in order to validate the method.

Vegetable oils are frequently applied in cosmetic products as emollient and moisturizing agents and in perfumery, they are mainly used as carriers for olfactive molecules. They form a category of natural and complex olfactive ingredients mixtures. The moisturizing of some of them was evaluated and compared to our reference, the mineral oil. The following are a list of suitable cosmetic agents: Almond oil, Mineral oil, Petroleum Jelly

### Example 5: Perfuming Compositions of the Invention

Compounds from different groups, described below as examples, were applied on the artificial skin model as it was already explained above and the TEWL was measured during 60 min. The following compositions were identified as having good moisturizing activity.

TABLE 1
Composition of model fragrances A, B and C

| | FRAGRANCE A | FRAGRANCE B | FRAGRANCE C |
|---|---|---|---|
| INGREDIENT | % | % | % |
| benzyl acetate | 8.0 | 21.7 | |
| (+-)-3,7-DIMETHYL-6-OCTEN-1-OL | 2.7 | | |
| (+-)-2,6-DIMETHYL-7-OCTEN-2-OL | 2.7 | 21.7 | |
| (+-)-TETRAHYDRO-2-ISOBUTYL-4- | 16.0 | | |
| METHYL-4(2H)-PYRANOL | | | |
| 4-CYCLOHEXYL-2-METHYL-2-BUTANOL | | 10.9 | |
| (E)-3,7-DIMETHYL-2,6-OCTADIEN-1-OL | 13.3 | | |
| DIPG | | | |
| 1-OXA-12-CYCLOHEXADECEN-2-ONE (A) + | 8.0 | | 20.0 |
| 1-OXA-13-CYCLOHEXADECEN-2-ONE | | | |
| (+)-METHYL (1S)-TRANS-3-OXO-2-PENTYL- | 13.3 | | 1 |
| 1-CYCLOPENTANE ACETATE | | | |
| (+)-2-{(1S)-1-[(1R)-3,3- | 5.3 | 4.3 | 6 |
| dimethylcyclohexyl]ethoxy}-2- | | | |
| methylpropyl propionate | | | |
| 1-((2RS,3RS)-2,3,8,8-tetramethyl- | 2.7 | 28.2 | |
| 1,2,3,4,5,6,7,8-octahydronaphthalen-2- | | | |
| yl)ethanone | | | |
| MUSCONE | | | |
| (+-)-(4E)-3-METHYL-4- | 1.3 | | 12 |
| CYCLOPENTADECEN-1-ONE | | | |
| 2-phenyl ethanol | 26.7 | | |
| CARYOPHYLENE | | | |
| (3aR,5aS,9aS,9bR)-3a,6,6,9a- | | 2.2 | 1 |
| tetramethyldodecahydronaphtho[2,1- | | | |
| b]furan | | | |
| oxacyclohexadecan-2-one | | 10.9 | 60 |
| 1-(5,5-dimethyl-1-cyclohexen-1-yl)-4- | | 0.1 | |
| penten-1-one | | | |
| | 100 | 100 | 100 |

### Essential Oils

Essential oils have been known and used for thousands of years due to their cosmetic and pharmaceutical benefits. They are natural mixtures of olfactive molecules and could be extracted from different parts of the plants such as roots, seeds, flowers, wood or fruit peels.

Four essential oils were chosen to prove the validity of our method: Lavender, Geranium, Rose damascena and Eucalyptus globulus EOs.

The moisturizing of these olfactive compositions was evaluated. Except the lavender EO, a versatile essential oil mainly known for its calming, relaxing and antiseptic properties, all the evaluated essential oils can be considered as moisturizing agents.

Geranium and Rose EOs have almost identical compositions and properties. Regarding their moisturizing potential, both oils can improve the skin hydration by water retention. Geranium EO is known for its regenerative effects on skin and balancing effects on sebum production. It also has antibacterial and anti-inflammatory properties. While soothing the skin, Geranium EO allows to keep moisture in thanks to its moisturizing effect. Regarding the Rose EO, it provides multiple benefits in addition to its valued fragrance, such as calming and anti-ageing properties. Thanks to its rich essential fatty acids content, this EO can improve the skin hydration levels more effectively than Geranium oil.

Eucalyptus EO is best known as antimicrobial agent, decongestant and insect repellent, but the results show that it can also decrease the water evaporation from the skin.

1. A method of using a composition comprising a fragrance component, wherein said fragrance component is present in an amount of from about 0.1% to 96% relative to the total weight of the composition, wherein the fragrance component comprises one or more perfume ingredients selected from the group consisting of: benzyl acetate, 1-oxa-12-cyclohexadecen-2-one (a)+1-oxa-13-cyclohexadecen-2-one, (+)-methyl (1s)-trans-3-oxo-2-pentyl-1-cycloheptaneacetate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one, (3ar,5as,9as,9br)-3a,6,6,9a-tetramethyldodecahydronaphtho[2,1-b]furan, oxacyclohexadecan-2-one and 1-(5,5-dimethyl-1-cyclohexen-1-yl)-4-penten-1-one, (+−)-8-methoxycedrane, 1-methyl-4-(1-methylethenyl)-cyclohexene, (3RS,3aRS,6SR,7aSR)-3,6-dimethylhexahydro-1-benzofuran-2(3H)-one (A)+(3RS,3aSR,6RS,7aRS)-3,6-dimethylhexahydro-1-benzofuran-2(3H)-one (B), (3aR,5aS,9aS,9bR)-3a,6,6,9a-tetramethyldodecahydronaphtho[2,1-b]furan, (+)-2-{(1S)-1-[(1R)-3,3-dimethylcyclohexyl]ethoxy}-2-methylpropyl propionate, almond oil, mineral oil, petroleum Jelly, wherein the method comprises using the composition as a skin moisturizer.

2. The method of claim 1, wherein the fragrance component comprises benzyl acetate, (+−)-3,7-dimethyl-6-octen-1-ol, (+−)-2,6-dimethyl-7-octen-2-ol (+−)-tetrahydro-2-isobutyl-4-methyl-4(2h)-pyranol, (e)-3,7-dimethyl-2,6-octadien-1-ol, 1-oxa-12-cyclohexadecen-2-one (a)+1-oxa-13-cyclohexadecen-2-one, (+)-methyl (1s)-trans-3-oxo-2-pentyl-1-cyclopentane acetate, (+)-2-{(1s)-1-[(1r)-3,3-dimethylcyclohexyl]ethoxy}-2-methylpropyl propionate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one and 2-phenyl ethanol.

3. The method of claim 1, wherein the fragrance component comprises benzyl acetate, (+−)-2,6-dimethyl-7-octen-2-ol, 4-cyclohexyl-2-methyl-2-butanol, (+)-2-{(1s)-1-[(1r)-3,3-dimethylcyclohexyl]ethoxy}-2-methylpropyl propionate, 1-((2rs,3rs)-2,3,8,8-tetramethyl-1,2,3,4,5,6,7,8-octahydronaphthalen-2-yl)ethenone, (3ar,5as,9as,9br)-3a,6,6,9a-tetramethyldodecahydronaphtho[2,1-b]furan, oxacyclohexadecan-2-one and 1-(5,5-dimethyl-1-cyclohexen-1-yl)-4-penten-1-one.

4. The method of claim 1, wherein the fragrance component comprises 1-oxa-12-cyclohexadecen-2-one (a)+1-oxa-13-cyclohexadecen-2-one, (+)-methyl (1s)-trans-3-oxo-2-pentyl-1-cyclopentane acetate, (+−)-(4e)-3-methyl-4-cyclopentadecen-1-one, 3ar,5as,9as,9br)-3a,6,6,9a-tetramethyldodecahydronaphtho[2,1-b]furan and oxacyclohexadecan-2-one.

5. The method of claim 1, wherein the composition further comprises a perfumery carrier and/or a perfuming co-ingredient and/or a perfumery adjuvant.

6. The method of claim 1, wherein the composition is included in a perfumery consumer product, wherein the method comprises using the perfumery consumer product as a skin moisturizer.

7. The method according to claim 6, wherein the perfumery consumer product is a perfume, a body-care product, a cosmetic preparation or a skin-care product.

8. The method according to claim 7, wherein the perfumery consumer product is a fine perfume, a splash or eau de parfum, a cologne, a shave or after-shave lotion, a shampoo, a hair conditioner, a coloring preparation, a color care product, a hair shaping product, a dental care product, a disinfectant, an intimate care product, a hair spray, a vanishing cream, a deodorant or antiperspirant, a hair remover, a tanning or sun product, a nail product, a skin cleansing, a makeup, a perfumed soap, a shower or bath mousse, an oil or a gel, a foot/hand care product, or a hygiene product.

9. A method to confer, enhance, improve or modify the skin moisturizing properties of a perfuming composition or of a perfumed article, which method comprises adding to said composition or article an effective amount of a composition comprising a fragrance component, wherein said fragrance component is present in an amount of from about 0.1% to 96% relative to the total weight of the composition, wherein the fragrance component comprises one or more perfume ingredients selected from the group consisting of: benzyl acetate, 1-oxa-12-cyclohexadecen-2-one (a)+1-oxa-13-cyclohexadecen-2-one, (+)-methyl (1s)-trans-3-oxo-2-pentyl-1-cycloheptaneacetate, 1-((2rs,3rs)-2,3,8,8-tetramethyl-1,2,3,4,5,6,7,8-octahydronaphthalen-2-yl)ethenone, (+−)-(4e)-3-methyl-4-cyclopentadecen-1-one, (3ar,5as,9as,9br)-3a,6,6,9a-tetramethyldodecahydronaphtho[2,1-b]furan, oxacyclohexadecan-2-one and 1-(5,5-dimethyl-1-cyclohexen-1-yl)-4-penten-1-one, (+−)-8-methoxycedrane, 1-methyl-4-(1-methylethenyl)-cyclohexene, (3RS,3aRS,6SR,7aSR)-3,6-dimethylhexahydro-1-benzofuran-2(3H)-one (A)+(3RS,3aSR,6RS,7aRS)-3,6-dimethylhexahydro-1-benzofuran-2(3H)-one (B), (3aR,5aS,9aS,9bR)-3a,6,6,9a-tetramethyldodecahydronaphtho[2,1-b]furan, (+)-2-{(1S)-1-[(1R)-3,3-dimethylcyclohexyl]ethoxy}-2-methylpropyl propionate, almond oil, mineral oil, petroleum Jelly.

10. The method of claim 6, wherein the method comprises applying an effective amount of the perfumery consumer product to the skin of a subject in need thereof, wherein the method confers, enhances, improves, or modifies skin moisture.

11. A skin replicating surface comprising (i) a hydrogen membrane layer comprising a water permeable substrate supporting a biopolymer, and (ii) a stratum corneum mimic layer.

12. The method of claim 1, wherein the method comprises applying an effective amount of the composition to the skin of a subject in need thereof, wherein the method confers, enhances, improves, or modifies skin moisture.
